(19) United States
(12) Patent Application Publication
(10) Pub. No.: US 2024/0245497 A1
(43) Pub. Date: Jul. 25, 2024

(54) PORTABLE SEDATION TOOTHBRUSH AND METHOD OF USE THEREOF

(71) Applicant: Gregory Robert Baker, Hazard, KY (US)

(72) Inventor: Gregory Robert Baker, Hazard, KY (US)

(21) Appl. No.: 18/625,671

(22) Filed: Apr. 3, 2024

(51) Int. Cl. A61C 19/08; A61C 17/00

(52) U.S. Cl. CPC A61C 19/08; A61C 17/00

(57) ABSTRACT

A portable sedation toothbrush with a brush head is proposed for surreptitiously administering low volumes of midazolam (or other similar sedative fluids) by entering into the oral cavity of non-cooperative patients just like any conventional toothbrush. The toothbrush is capable of drawing in any sedative fluid from a glass vial and piercing its rubber cap. The proposed toothbrush is capable of gradually dripping an effective volume of 2.5 ml (for example) sedative fluid through sublingual administration. The sedative fluid comes out of a nozzle situated at the top of brush head housing and is aimed at being deposited at the underside of the tongue. The toothbrush is capable of being operated by a single medical professional.

### TECHNICAL FIELD

The present disclosure relates to toothbrushes, in particular to a portable sedation toothbrush configured for surreptitious and sublingual delivery of low volume of midazolam (or other similar sedative fluids) to carry out the anesthetic procedure in non-cooperative or non-accepting patients to facilitate successive dental procedures (E.g. surgery).

### BACKGROUND

A lot of patients with intellectual disabilities, for example with autism or similar disorders, or patients who are fearful or overly alert may have difficulty accepting premedication with existing methods of delivery. In such patients undergoing dental treatment, delivering premedication in the form of midazolam (or other similar sedative fluids) utilizing traditional methods of delivery is a big challenge due to their non-acceptance nature. Several attempts to promote dental treatment acceptance through desensitization and other means have been made however in many instances are proven to be unsuccessful in prompting the use of sedation or general anesthesia for the safe completion of dental treatment. The common procedures for anesthesia delivery, such as intravenous (IV) cannulation or mask fitting for anesthetics inhalation, have proven to be very stressful in such patients. If the patient is unable to cooperate, physical restraint may be necessary, potentially leading to problematic psychological and/or physical insult.

In recent research titled “Successful Premedication with Sublingual Midazolam Using a Suction Toothbrush,” by Jun Hirokawa, and Naomi Kimata, published online 2023 Jun. 28, the use of a suction toothbrush for sublingual administration of midazolam is disclosed. As presented in the paper, the administration of sublingual midazolam as premedication is done using a suction toothbrush while brushing the patient's teeth.

As can be seen in FIG. 1, in this method, a suction toothbrush 11 is connected to a syringe 13 using a long tube/hose 12 with two connectors 12a, and 12b provided at two ends. The syringe 13 is initially connected with a needle for drawing in a sedation fluid by piercing the cap of a sedation fluid bottle. Once the sedation fluid is filled in the syringe 13 the needle is removed and the tip of syringe 13 is connected to the toothbrush through the tube/hose 12.

In operation, as seen in FIG. 2, the patient is kept unobservant of the hose 12 and the syringe 13 kept inside a pocket of the care provider's clothing. To elaborate, utilizing this device requires the involvement of two persons, one being a doctor 15 holding the suction toothbrush 11 connected to the syringe 13 filled with the sedative file and held by another doctor or care provider 25. During the premedication delivery of the sedation fluid, the syringe 13 is remotely plunged by the care provider 25 holding the syringe in his pocket. The fluid travels through the hose and gets disposed of by sublingual administration during toothbrushing by the doctor 15.

Although the above-discussed method is found to be successful in sedating the patient and making them feel comfortable to accept the face mask for inhalational induction without discomfort, allowing the dental treatment to be completed under IVS, it suffers from a number of shortcomings, for example, the device is difficult to use as the hose can get tangled and draw the obvious attention of the fearful patient. Further, the long hose connection dangling from the toothbrush, and the caregivers hand placed inside the pocket raises curiosity and eventual suspicion in patients. This can lead to potential resistance to anesthesia, thus making the successive steps (for example surgery) for the dental treatment in patients very difficult. Further, the doctor has to put on and take off the needle to load the sedative fluid into the syringe and then deliver the same to the patient, which may be time-consuming task.

In the light of the aforementioned background, the inventor herein is proposing a more reliance, and productive design for a portable sedation toothbrush that do not require any separate hose that can attract unnecessary attention of the fearful patient, or require involvement of multiple caregivers for sublingual administration of midazolam (or other similar sedative fluids), and/or require putting on and taking off needle to load the sedative fluid and delivering the same to the patient.

What is desired is a more simplified and result-oriented solution that may not attract the attention of fearful patients and yet be very easy for carrying out sublingual administration of low volumes of midazolam (or other similar sedative fluids) in non-cooperative or non-accepting patients to help doctors in carrying out any successive dental medical procedure.

### BRIEF SUMMARY

Accordingly, it is an object of the present invention to provide a portable sedation toothbrush that's capable of being operated by a single user (doctor) and do not require involvement of multiple caregiver persons as described in the prior art device discussed in the background section.

The proposed sedation toothbrush design aims at carrying out anesthetic procedure more conveniently in the fearful patients making the successive steps (for example surgery) for the dental treatment in patients very easy.

The proposed sedation toothbrush design aims at saving the time of doctors who have to spend considerable time in putting on and take off the needle of syringe to load the sedative fluid into the syringe and then deliver the same to the patient orally.

Embodiments of the present invention discloses a portable sedation toothbrush (30). The sedation toothbrush includes a longitudinally extended hollow handle portion (33) with a gripping section (33a) and defining an internal handle chamber (330 for selectively holding a predefined quantity of a sedative fluid therein. The handle portion (33) having a tip (33g) at a first end for receiving a needle (33d), and a plunger (33b) fitted at a second end selectively movable in a first direction, or a second direction. The plunger (33b) is moved in the first direction to draw in the sedative fluid into the internal handle chamber (330 of the handle portion (33) when the needle (33d) is in contact with the sedative fluid, and the plunger (33b) is moved in the second direction to dispense the sedative fluid out of the needle (33d).

The sedation toothbrush further includes a longitudinally extended hollow head portion (32) removably attachable to the handle portion (33). The head portion (32) defines a head housing (32b), and a neck section (32a) extending from the head housing (32b). The head housing (32b) having bristles(32c) extending therethrough, and a nozzle (32d) configured thereon for sublingual administration of the sedative fluid dispensed out of the needle (33d) when the plunder (33b) is moved in the second direction.

Various advantages and features of the present invention are described herein with specificity so as to make the present invention understandable to one of ordinary skill in the art, both with respect to how to practice the present invention and how to make the present invention.

### BRIEF DESCRIPTION OF THE DRAWINGS

The above set forth and other features of the present invention are made more apparent in the ensuing description of the preferred embodiments when read in conjunction with the attached drawings, wherein:

FIGS. 1 and 2 show a prior art suction toothbrush system and method of delivering midazolam utilizing the suction toothbrush system through sublingual administration in non-cooperative patients.

FIG. 3 shows a front perspective view of a portable sedation toothbrush for surreptitiously delivering low volumes of midazolam (or other similar sedative fluids) through sublingual administration in non-cooperative patients, according to an embodiment of the present invention.

FIG. 4 shows an exploded view of the portable sedation toothbrush of FIG. 3.

FIG. 5 shows a cross-sectional view of the portable sedation toothbrush of FIG. 3 in an unusable state with the plunger in a completely plunged in position.

FIG. 6 shows a cross-sectional view of the portable sedation toothbrush of FIG. 3 in a usable state with the plunger in a pulled-out position.

FIG. 7 shows the portable sedation toothbrush of FIG. 3 with the interior of the brush made visible.

FIG. 8 shows a handle portion of the sedation toothbrush of FIG. 4 with the plunger in a pulled-out position.

FIGS. 9-12 show a method of using the portable sedation toothbrush of FIG. 3 for surreptitiously delivering low volumes of midazolam (or other similar sedative fluids) through sublingual administration in non-cooperative patients, according to an embodiment of the present invention.

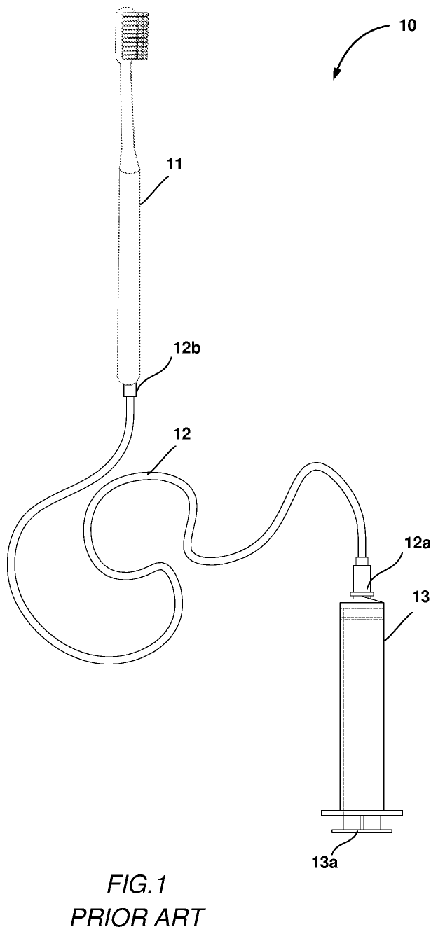

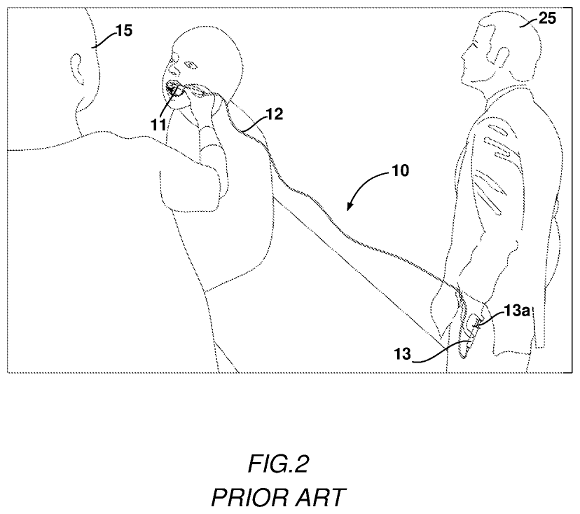

Identical reference numerals in the figures are intended to indicate like parts, although not every feature in every figure may be called out with a reference numeral.

### DETAILED DESCRIPTION

Certain terminology is used in the following description for reference only and is not limiting. Unless specifically set forth herein, the terms “a,” “an,” and “the” are not limited to one element, but instead should be read as meaning “at least one.” The terminology includes the words noted above, derivatives thereof, and words of similar import. Before describing the present invention in detail, it should be observed that the present invention utilizes a combination of components, which constitutes an electric toothbrush system and associated components thereof. Accordingly, the components have been represented, showing only specific details that are pertinent for an understanding of the present invention so as not to obscure the disclosure with details that may be readily apparent to those with ordinary skill in the art having the benefit of the description herein. As required, the detailed embodiments of the present invention are disclosed herein. However, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

The words “comprising”, “having”, “containing”, and “including”, and other forms thereof, are intended to be equivalent in meaning and be open-ended in that an item or items following any one of these words is not meant to be an exhaustive listing of such item or items or meant to be limited to only the listed item or items. In the context of the present invention, the terms “doctor,”, “caregiver,”, “user,” and so on are all interchangeably used.

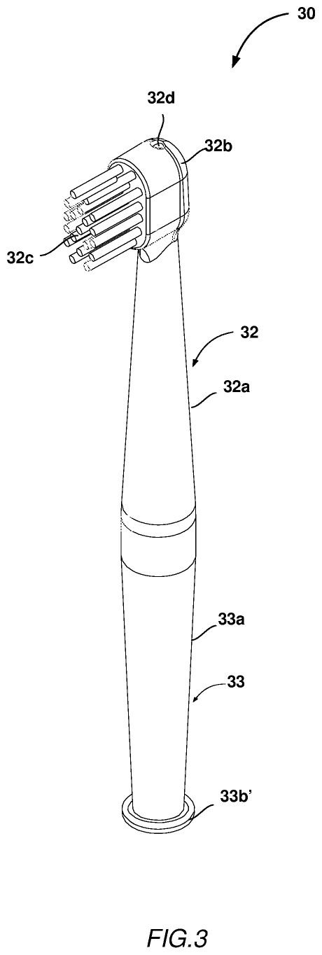

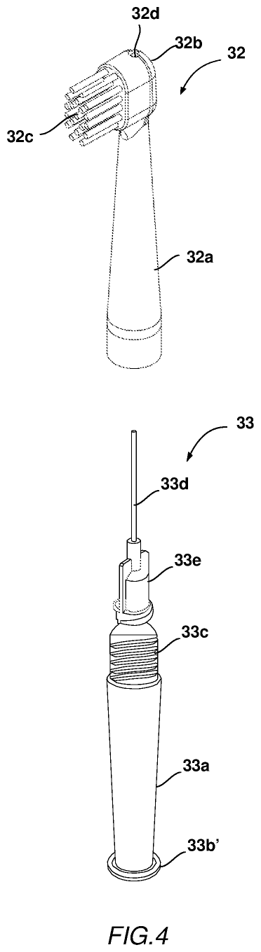

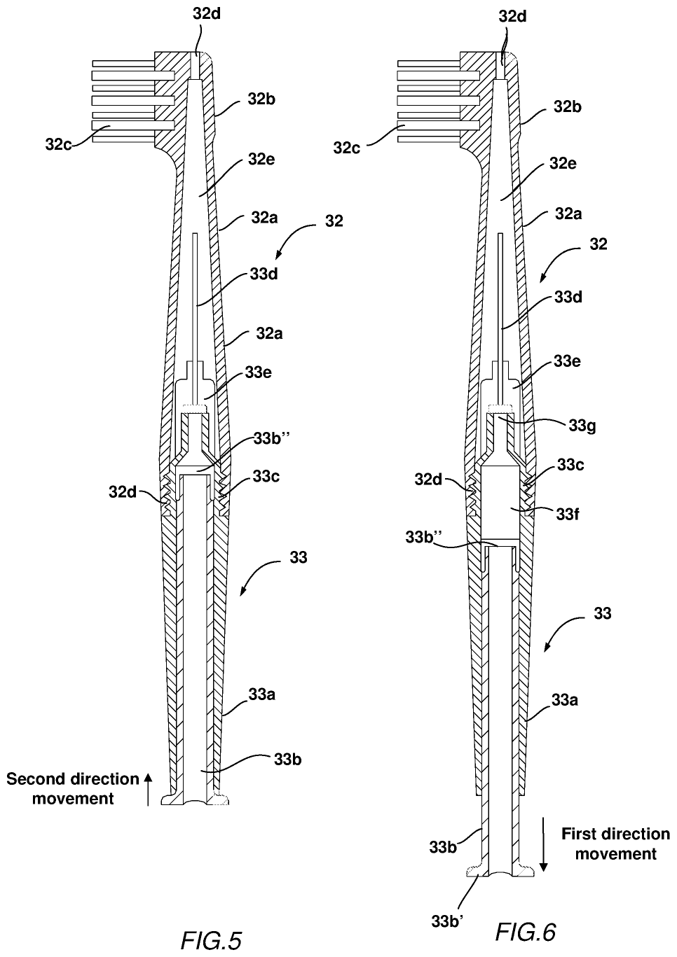

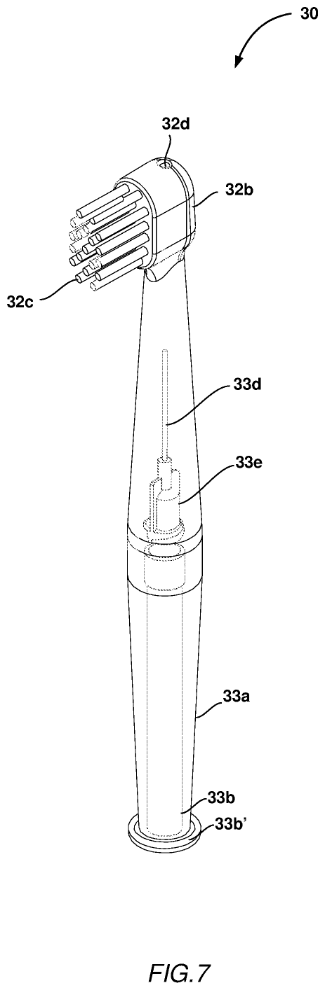

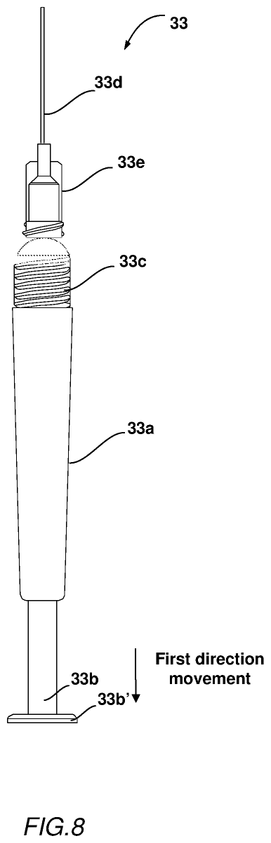

Referring to FIGS. 3-12, there is provided a portable sedation toothbrush and an associated method of using the portable sedation toothbrush for surreptitious and sublingual delivery of low volume of midazolam (or other similar sedative fluids) in non-cooperative or non-accepting patients to facilitate successive dental procedures. Referring particularly to FIGS. 3-8, the portable sedation toothbrush 30 includes a longitudinally extended hollow handle portion 33. The handle portion 33 includes a gripping section 33a used for gripping the toothbrush 30 when in use. The handle portion 33 further defines an internal handle chamber 33f for selectively holding a predefined quantity of sedative fluid inside. The sedative fluid may include but is not limited to midazolam.

Further, the handle portion 33 includes a tip 33g at one end for receiving a needle 33d, and a plunger 33b fitted on the other end. The needle 33d is fixedly connected to the tip 33g of the handle portion 33 according to an embodiment. According to another embodiment, the needle 33d may be removably connected to the tip 33g of the handle portion 33. In an embodiment, the needle 33d may be connected to the tip 33g using a connector 33e. In some other embodiment, the needle 33d may be directly connected to the tip 33g. The plunger 33b is selectively movable in a first direction (plunged out position), or a second direction (plunged in position). In an embodiment, the plunger 33b comprises a flat base 33b′ at one end to allow the portable sedation toothbrush 30 to stand on flat surfaces while the other end of the plunger 33b has a rubber cap 33b″ that provides a degree of interference inside the gripping section 33a of the handle portion 33 to facilitate water sealing, create vacuum pressure and produce capillary effect along the conical hollow gripping section 33a. In some other embodiment, the flat base 33b′ may not be present.

Furthermore, the handle portion 33 includes a set of threads 33c externally configured on the handle portion 33. The threads 33c may be helical or configured in other configurations/shapes. The handle portion 33 is preferably made reusable using suitable materials known in the art such as plastic, ABS, wood, and so on.

In operation, the plunger 33b is moved in the first direction (plunged out position) to draw in the sedative fluid into the internal handle chamber 33f of the handle portion 33 when the needle 33d comes in contact with the sedative fluid and the plunger 33b is moved in the second direction or plunged out position to dispense the sedative fluid out of the needle 33d.

Further, according to an embodiment, the toothbrush 30 includes a longitudinally extended hollow head portion 32 removably attachable to the handle portion 33. The head portion 32 according to a preferred embodiment is made of disposable material such as paper or other non-hazardous material. In an embodiment, the head portion 32 is threadingly engageable to the handle portion 33 when the head portion 32 is disposed over the handle portion 33. To facilitate this, the head portion 32 includes a set of threads 32d internally configured within the neck section 32a. The threads 32d may be helical or configured in other configurations/shapes. In assembly, when the head portion 32 is removably attached to the handle portion 33, the tip 33g, the needle 33d, and a portion of the internal handle chamber 33f of the handle portion 33 is received within a hollow space 32e of the head portion 32. The hollow space 32e of the head portion 32 acts as a passageway for the sedative fluid dispensed out of the needle 33d to reach the nozzle 32d located on the head housing 32b. The hollow space 32e is in the order of decreasing diameter along the length extending from the neck section 32a to the head housing 32b (similar to conical shape).

According to an embodiment, the head portion 32 defines a head housing 32b, and a neck section 32a extending from the head housing 32b. The head housing 32b includes bristles 32c extending therethrough. The head housing 32b also includes a nozzle 32d configured thereon for sublingual administration of the sedative fluid dispensed out of the needle 33d when the plunger 33b is moved in the second direction. The nozzle 32d may be made of plastic or any other suitable material and includes about 0.2 mm diameter orifice. In an embodiment, the nozzle is disposed of at the top of the head housing 32b.

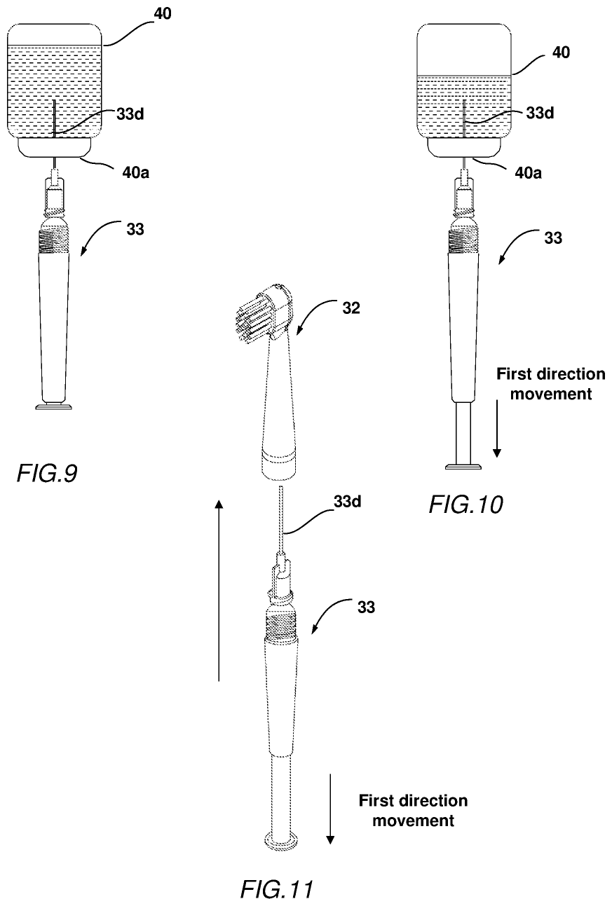

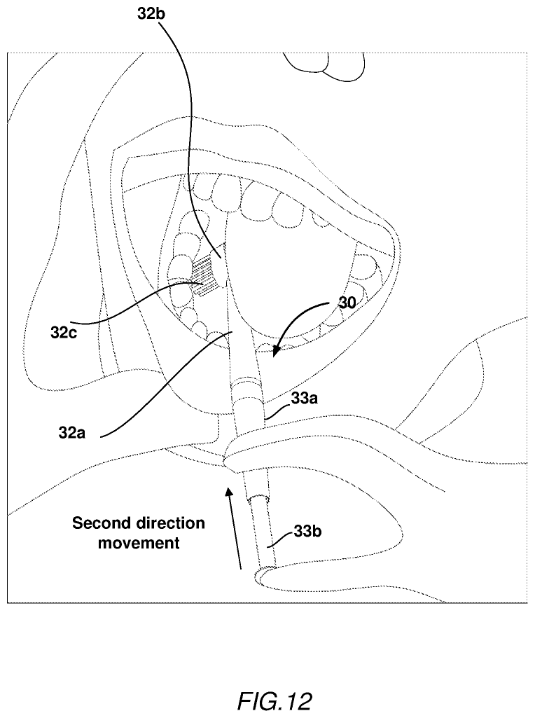

Now referring to FIGS. 9-12 that shows a method of using the portable sedation toothbrush described above for surreptitiously delivering low volumes of midazolam (or other similar sedative fluids) through sublingual administration in non-cooperative patients, according to an embodiment of the present invention. FIGS. 9 and 10 together show the process of filling sedation fluid into the handle portion 33 of the toothbrush 30. FIG. 9 shows the piercing of a vial 40 Like the most common clinician practice, a user (doctor or other medical professional) can pierce a rubber cap 40a of the medicinal vial 40 first utilizing the needle 33d of the handle portion 33. The rubber cap 40a is pierced with a pointy end of the needle 33d to facilitate the handle portion 33 to draw the fluid inside the chamber 33f. FIG. 10 shows the plunging out or moving the plunger in the first direction. The first direction (plunged out position) of the plunger 33b is to draw in the sedative fluid into the internal handle chamber 33f of the handle portion 33 when a pointy end of the needle 33d remains inserted in the vial 40. Once the handle portion 33 is loaded with the fluid, the user or medical professional can dispose of the empty vial in a biomedical waste bin or other appropriate places. FIG. 11 shows the engagement process of the head portion 32 with the handle portion 33 to form the proposed toothbrush 30 ready for being used to surreptitiously carry out sublingual administration of the sedative fluid through the nozzle 32d while brushing the teeth of a patient preferably seen in FIG. 12. The doctor can use the toothbrush 30 to clean the oral cavity of the patient with an aromatic toothpaste (just like any conventional brushing) while squirting small amounts of the fluid sublingually to mask the smell and also orally inject the sedative fluid into the patient's body. By sublingual route, immediate absorption of the drug takes place resulting in an immediate effect too.

The handle portion 33 and the head portion 32 may be configured in different shapes and sizes. Further, the handle portion 33 and the head portion 32 may be made utilizing several materials however, it is preferred that the head portion 32 is made of disposable material and the handle portion 33 is made utilizing reusable material. Another big advantage of the present invention is to provide a sedation toothbrush with interchangeable head portion 32 that makes the product more acceptable to the public from a safety and cost point of view.

While the invention has been described in connection with what is presently considered to be the most practical and preferred embodiments, it is to be understood that the invention is not to be limited to the disclosed embodiments, but, on the contrary, is intended to cover various modifications and equivalent arrangements included within the spirit and scope of the appended claims.

What is claimed is:

1. A portable sedation toothbrush (30), comprising:
a longitudinally extended hollow handle portion (33) with a gripping section (33a) and defining an internal handle chamber (33f) for selectively holding a predefined quantity of a sedative fluid therein, the handle portion (33) having a tip (33g) at a first end for receiving a needle (33d), and a plunger (33b) fitted at a second end selectively movable in a first direction, or a second direction;
wherein, the plunger (33b) is moved in the first direction to draw in the sedative fluid into the internal handle chamber (33f) of the handle portion (33) when the needle (33d) is in contact with the sedative fluid, and the plunger (33b) is moved in the second direction to dispense the sedative fluid out of the needle (33d); and
a longitudinally extended hollow head portion (32) removably attachable to the handle portion (33), wherein the head portion (32) defines a head housing (32b), and a neck section (32a) extending from the head housing (32b); the head housing (32b) having bristles (32c) extending therethrough, and a nozzle (32d) configured thereon for sublingual administration of the sedative fluid dispensed out of the needle (33d) when the plunder (33b) is moved in the second direction.

2. The portable sedation toothbrush (30) of claim 1, wherein the needle (33d) is connected to the tip (33g) of the handle portion (33) using a connector (33e).

3. The portable sedation toothbrush (30) of claim 1, wherein the sedative fluid is midazolam.

4. The portable sedation toothbrush (30) of claim 1, wherein the head portion (32) comprises a first set of threads (32d) internally configured within the neck section (32a).

5. The portable sedation toothbrush (30) of claim 1, wherein the handle portion (33) comprises a second set of threads (33c) externally configured on the handle portion (33).

6. The portable sedation toothbrush (30) of claim 2, wherein the needle (33d) is fixedly or removably connected to the tip (33g) of the handle portion (33) using the connector (33e).

7. The portable sedation toothbrush (30) of claim 1, wherein the head portion (32) is threadingly engageable to the handle portion (33) when the head portion (32) is disposed over the handle portion (33).

8. The portable sedation toothbrush (30) of claim 1, wherein the head portion (32) is disposable.

9. The portable sedation toothbrush (30) of claim 1, wherein the handle portion (33) is reusable.

10. The portable sedation toothbrush (30) of claim 1, wherein the tip (33g), the needle (33d), and a portion of the internal handle chamber (33f) of the handle portion (33) is received within a hollow space (32e) of the head portion (32) when the head portion (32) is removably attached to the handle portion (33).

11. The portable sedation toothbrush (30) of claim 1, wherein the handle portion (33) is disengaged from the head portion (32) to fill in the sedative fluid into the handle chamber (33f) of the handle portion (33) by moving the plunger (33b) in the first direction.

12. The portable sedation toothbrush (30) of claim 1, wherein the handle portion (33) is engaged to the head portion (32) to surreptitiously carry out sublingual administration of the sedative fluid through the nozzle (32d) while brushing the teeth of a patient utilizing the portable sedation toothbrush (30).

13. The portable sedation toothbrush (30) of claim 10, wherein the hollow space (32e) of the head portion (32) acts as a passageway for the sedative fluid dispensed out of the needle (33d) to reach the nozzle (32d).

14. The portable sedation toothbrush (30) of claim 13, wherein the hollow space (32e) within the head portion (32) acting as the passageway for the sedative fluid is in the order of decreasing diameter along the length extending from the neck section (32a) to the head housing (32b).

15. The portable sedation toothbrush (30) of claim 1, wherein the nozzle (32d) comprises about 0.2 mm diameter orifice.

16. The portable sedation toothbrush (30) of claim 1, wherein the plunger (33b) comprises a flat base (33b′) at one end allowing the portable sedation toothbrush (30) to stand on flat surfaces while the other end has a rubber cap (33b″) that provides a degree of interference inside the gripping section (33a) of the handle portion (33) to facilitate water sealing, create vacuum pressure and produce capillary effect along the conical hollow gripping section (33a).
